(19) United States
(12) Patent Application Publication
(10) Pub. No.: US 2024/0335460 A1
(43) Pub. Date: Oct. 10, 2024

(54) PROCESS FOR PREPARING CANNABINOID-CONTAINING PARTICLES

(71) Applicant: STABICAN B.V., Almere (NL)

(72) Inventor: Robin DE BRUIJN, Eindhoven (NL)

(21) Appl. No.: 18/578,646

(22) PCT Filed: Jul. 12, 2022
(86) PCT No.: PCT/NL2022/050403

§ 371 Date: Jan. 11, 2024

(30) Foreign Application Priority Data
Jul. 13, 2021 (NL) 2028708

(51) Int. Cl. A61K 31/00; A61K 9/00; A61K 9/20; A61K 9/50

(52) U.S. Cl. CPC A61K 31/658; A61K 9/0002; A61K 9/2081; A61K 9/5089

(57) ABSTRACT

The invention relates to a process for preparing cannabinoid-containing particles, wherein first a solution is provided wherein the solvent has a water solubility in the range of 2-100 g/L at 25° C. and wherein the solution comprises 1) a cannabinoid component comprising one or more cannabinoids; and 2) a shell-forming component comprising one or more compounds selected from the group of C10-C30 fatty alcohols. C10-C30 fatty acids and esters of C10-C30 fatty alcohols and C10-C30 fatty acids. Then, droplets of the solution are generated in an aqueous medium and the solvent is allowed to migrate from the droplets to the aqueous medium to thereby form solid particles wherein a shell of shell-forming component encapsulates the cannabinoid component.

### FIELD OF THE INVENTION

The present invention relates to a process for preparing cannabinoid-containing particles, to a particle obtainable by such method, to a cannabinoid-containing particle and to such particle for medical use.

### BACKGROUND

Cannabis plants produce a group of chemicals called cannabinoids, which are terpenophenolic compounds with a common structural motif. They differ mainly in the way their common precursor cannabigerol is cyclized. Cannabinoids in the form of cannabis and extracts thereof have been used for centuries for both medicinal and recreational purposes. They are attractive due to their particular psychoactive and physical effects.

More than hundred cannabinoids have been identified in Cannabis plants, of which the most prevalent are delta-9-tetrahydrocannabinol (THC), cannabidiol (CBD), and cannabinol (CBN). THC is the primary psychoactive component of the plant and has also found use in the treatment of a wide range of medical conditions. More specifically, THC binds to specific receptors in the brain called cannabinoid receptors and, in doing so, causes pain reduction, may reduce aggression, can stimulate appetite, and helps reduce nausea.

The widely known recreational use of cannabis involves the inhalation of smoke of burning cannabis plant parts, but many patients and other consumers of cannabis prefer to administer cannabis by eating or drinking it, rather than smoking. Moreover, especially for medical purposes, it is important that an exact dosage of one or more intended cannabinoids can be administered, which can hardly be achieved by smoking.

Oral dosage forms of cannabinoids are a logical alternative for smoking. However, despite many research and development efforts, the oral dosage forms known to date still suffer from serious shortcomings. It is difficult to prepare them in a reproducible way, in particular to include the same amounts of cannabinoid(s) in each dosage composition.

Further, the stability of conventional dosage forms is often insufficient, which is in particular encountered when it is aimed to use the composition as a pharmaceutical base product for the preparation of various kinds of cannabinoid-based medicine since this requires long shelf lives. Another disadvantage is that many conventional dosage forms are difficult to handle as a substance, which is in particular caused by their tackyness. This is for example the case when THC is present, because the oily nature of THC results in a high tackiness of compositions comprising THC. Many other cannabinoids and mixtures of cannabinoids are also present as an oil. This detrimental property severely thwarts the manufacturing of cannabinoid dosage forms in the form of a powder in an easy and reliable way.

In particular microparticles and nanoparticles where the oily and/or tacky cannabinoid is suspended in a filler material (e.g. a wax) suffer from tackiness and exhibit a short shelf-life. This is because a significant portion of cannabinoids in such compositions resides at the interface with air. Thus, such compositions provide no true and durable encapsulation of a cannabinoid. Such setting wherein oily and/or tacky cannabinoids are exposed to the surrounding atmosphere of the material not only makes the material as such oily and tacky, but obviously also limits the long-term stability of the cannabinoids that are suspended in the material.

A particular type of cannabinoid dosage form concerns those comprising the so-called whole-plant cannabis extracts. Such mixture of many known and unknown components is interesting from a medicinal point of view, but the reproducible preparation of stable and easy-to-handle formulations thereof remains a big challenge.

It can be generally stated that at present, dosages of cannabinoids are not available in a satisfactory form, e.g. a form that is non-tacky and/or has a long shelf-life. In particular, there is a need for a standardized formulation comprising one or more particular cannabinoids, preferably in a powder form, as a way to enable consumers and patients to accurately and repeatably take the same dose of cannabinoid to address their (medical) needs. More in particular, there is a need for a pharmaceutical base product that can serve as a standard for the preparation of cannabinoid-based medicine.

At the same time, it is desired that the manufacturing of such product can be performed in a way that is easy, reliable and/or reproducible. It is in particular desired that there is a manufacturing method that does not yield a tacky product, or at least that in the manufacturing method there is control over the tackiness of the product.

### SUMMARY OF THE INVENTION

It is therefore an object of the present invention to provide a formulation comprising a cannabinoid that does not suffer from one or more of the abovementioned shortcomings.

It has now been found that a particular encapsulation method may overcome these shortcomings.

Accordingly, the invention relates to a process for preparing cannabinoid-containing particles, comprising
providing a solution wherein the following components are dissolved in a solvent that has a water solubility in the range of 2-100 g/L at 25° C.:
   a cannabinoid component comprising one or more cannabinoids;
   a shell-forming component comprising one or more compounds selected from the group of C10-C30 fatty alcohols, C10-C30 fatty acids and esters of C10-C30 fatty alcohols and C10-C30 fatty acids;
generating droplets of the solution in an aqueous medium and allowing the solvent to migrate from the droplets to the aqueous medium to thereby form solid particles wherein a shell of shell-forming component encapsulates the cannabinoid component.

The invention also relates to a cannabinoid-containing particle obtainable by such process.

The invention also relates to a cannabinoid-containing particle wherein
one or more cannabinoids are encapsulated by a shell of one or more compounds selected from the group of C10-C30 fatty alcohols, C10-C30 fatty acids and esters of C10-C30 fatty alcohols and C10-C30 fatty acids;
the content of cannabinoid in the particle is in the range of 25-95 wt. %.

The invention also relates to a pharmaceutical composition comprising such a cannabinoid-containing particle and a pharmaceutically acceptable excipient.

The invention also relates to a method for treating a medical condition of a human or an animal, comprising administering to the human or animal an effective amount of such a cannabinoid-containing particle or such a pharmaceutical composition, wherein the medical condition is selected from the group of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

### BRIEF DESCRIPTION OF THE DRAWINGS

FIG. 1 displays a micrograph of particles containing THC that are produced with a method of the invention.

FIG. 2 displays a micrograph of particles containing CBD that are produced with a method of the invention.

FIG. 3 displays an enlargement of a part of FIG. 2.

### DETAILED DESCRIPTION OF THE INVENTION

The solvent has a solubility in water that is in the range of 2-100 g/L at 25° C. This range on the one hand ensures that the solution of the cannabinoid component and the shell-forming component in the solvent is capable of existing as droplets in a water phase; and on the other hand that the solvent migrates from the droplet to the water phase. For example, a solvent with a solubility higher than 100 g/L (including infinite solubility), will not form droplets, or only droplets with insufficient stability, so that the two water-insoluble components do not phase separate and solid particles are not formed. Also, a solvent with a solubility lower than 2 g/L (including complete insolubility) will nevertheless form droplets, but its migration from the droplets to the water phase, if any, cannot be accomplished under satisfying conditions.

The solubility of the solvent in water may also be in the range of 6-90 g/L at 25° C., in the range of 8-80 g/L at 25° C., in the range of 10-70 g/L at 25° C., in the range of 12-60 g/L at 25° C., or in the range of 15-50 g/L at 25° C. It may also be in the range of 5-75 g/L at 25° C., in the range of 8-50 g/L at 25° C. or in the range of 10-40 g/L at 25° C.

The solvent may be selected from the group of benzyl alcohol, 1-butanol, n-butyl acetate, gamma-butyrolacton, chloroform, 1,2-dichloroethane, diethylene glycol, diethyl ether, diethoxyethane, di-isopropylether, ethyl acetate, methyl t-butyl ether, methylene chloride, N-methyl-2-pyrrolidinone, nitromethane, 1-pentanol, 2-pentanol, 3-pentanol, 3-pentanone, benzaldehyde, prenol, o-cresol, m-cresol, and p-cresol. The solvent may also be a terpenoid with a water solubility in the range of 2-100 g/L at 25° C. In particular, the solvent is benzyl alcohol or methylene chloride.

The solvent usually has a molar mass of less than 200 g/mol. It is in particular less than 150 g/mol. It may also be less than 140 g/mol, less than 125 g/mol or less than 100 g/mol.

Usually, the log P_{oct/wat} of the solvent is lower than the P_{oct/wat} of each of the one or more cannabinoids in the cannabinoid component. Herein, P is the partition coefficient, which is defined as a particular ratio of the concentrations of a solute between 1-octanol and water (a biphase of two liquid phases). A higher value of P indicates a higher lipophilicity than a lower value of P does. In case of the present invention, the solute is the solvent that is used to provide the solution wherein the shell-forming component and the cannabinoid component are dissolved. When the log P_{oct/wat} of the solvent is lower than the P_{oct/wat} of a cannabinoid, then the solvent is more prone to migration from the droplet to the water than the cannabinoid is.

The solution in a process of the invention comprises a mixture of the two components that in the end make up the cannabinoid-containing particles. This means that both components are dissolved in the solvent. The migration of the solvent from the droplet to the aqueous medium has the effect that both components come separately out of solution and gain their natural appearance, i.e. the appearance they normally have as a neat substance; the C10-C30 fatty acids, alcohols and ester are solid (or at least waxy), while the cannabinoid is a liquid or an oil, such as THC. Importantly, the solvent migration also results in the separation of both components, wherein the cannabinoid component constitutes the inner part of the particles and the shell-forming component constitutes a shell that completely surrounds the cannabinoid component.

Usually, the solution consists of these two components and the solvent, i.e. no other dissolved or undissolved substances are present in the solution. It is however possible that certain additives are contained in the solution (preferably dissolved therein), which either end up in the produced particle or migrate together with the solvent. For example, one or more additional active pharmaceutical ingredients other than cannabinoid(s) may be present. Also, other extractives from the Cannabis plant may be present, especially when the cannabinoid component comprises a whole-plant cannabis extract. It is also possible that a co-solvent is present in the solution, which migrates together with the solvent (i.e. the primary solvent) out of the droplet (or particle) into the aqueous medium. For example, such co-solvent is a solvent selected from the group of the (primary) solvents mentioned above.

In the solution, the weight ratio of cannabinoid component to shell-forming component is usually in the range of 0.05:0.95 to 0.95:0.05. The lower this ratio, the thicker the shell and the less cannabinoid is encapsulated by the shell (in relative terms).

It was observed that the tackiness/dryness of the particles can be tuned by changing the weight ratio of cannabinoid component to shell-forming component in the solution; a lower ratio appears to yield dryer particles (less tacky). It is contemplated that migration of the solvent is accompanied by the migration of minor amounts of cannabinoid that subsequently get stuck in the shell during formation of the particles, be it close to the shell's interface with the cannabinoid or further towards its interface with the outside environment. When the particles contain a minor amount of delta-9-tetrahydrocannabinol in their shell, it is envisioned that they become more tacky due to the tacky nature of delta-9-tetrahydrocannabinol. By applying a solution with a lower weight ratio of cannabinoid component to shell-forming component, the level of cannabinoid incorporated in the material of the shell itself will be decreased, yielding particles of a dry nature. Following this methodology, it was even possible to obtain the particles in the form of a free flowing powder.

It is thus advantageous when the weight ratio of cannabinoid component to shell-forming component in the solution is low. On the other hand, however, such low ratio means that the weight percentage of cannabinoid in the final particle is low, and thus also in the entire formulation. This is not always desired. Therefore, the weight ratio of cannabinoid component to shell-forming component in the solution in a process of the invention is a balance between tackiness and cannabinoid content of the product. Preferably, therefore, the ratio is in the range of 0.25:0.75 to 0.95:0.05, in particular in the range of 0.50:0.50 to 0.90:0.10. It may also be in the range of 0.40:0.60 to 0.80:0.20, or in the range of 0.60:0.40 to 0.95:0.05.

Thus, a process of the invention allows to prepare particles in an aqueous suspension that are not tacky at all. Generally, this is achieved when the shell-forming component is present in an amount of at least 30 wt. % (weight ratio of cannabinoid component to shell-forming component in the solution is at least 30:70). This for example allows the preparation of a dry and non-tacky delta-9-tetrahydrocannabinol (THC) formulation. This is unprecedented in the art, since no solution has yet been found to the problem of tackiness of THC-containing compositions—a problem that has dominated the field of cannabinoid encapsulation for decades.

In addition, the cannabinoids that are contained in the particles thus formed are surprisingly stable. The non-tackiness and the high stability allow the preparation of a cannabinoid-containing formulation that may serve as a pharmaceutical base product, which is a long-felt need in the medical field.

Another important property of the process of the invention is the encapsulation efficiency, which is the fraction of initially present cannabinoid that has been encapsulated by the process. This was determined for a process wherein THC was the only cannabinoid present and wherein benzyl alcohol was used as the solvent (as is further elaborated in the Examples' section 4). It appeared that it is possible to encapsulate 85 wt. % of the THC that was added in the process, and that the resulting product was a dry, non-tacky solid. CBD as well as a mixture of THC and CBD could also be encapsulated with a comparable efficiency (see Examples).

It may however well be that this 85% merely reflects the yield of the isolation process of the particles (e.g. of the filtration and washing procedures), and that it forms a minimum value for the encapsulation efficiency. The actual encapsulation efficiency may even be close to 100%, which is supported by the observation that no cannabinoid could be identified in the hardware used in the process (such as a beaker or funnel) and in the process fluids that were disposed (such as filtration liquids or washing liquids). Moreover, when filter residue was analysed, the ratio of cannabinoid component to shell-forming component was identical to that of the starting materials of the process.

Another advantageous property of the process of the invention is that cannabinoid levels in the particles of over 70 wt. % can be achieved (see Examples' section 5 and 13). This high encapsulation percentage as such is already a big step forward, but it is all the more a great advantage that this has been achieved by losing only 15 wt. % of the initially present cannabinoid (i.e. the cannabinoid present at the start of the encapsulation procedure). This concerns the encapsulation efficiency of 85% as discussed above.

In conventional encapsulations, it is often seen that in order to achieve a high ratio of encapsulated compound to encapsulant in the product, the initial ratio of both components has to be much higher to account for the many losses during the encapsulation process. In the present invention, however, the encapsulation of high amounts of cannabinoid does not go hand in hand with large losses of cannabinoid. Thus, it is an advantage of the present invention that a high cannabinoid weight percentage in the particles is realized in combination with a high encapsulation efficiency.

The process of the invention further demonstrates that the encapsulation efficiencies are remarkably constant for different runs. The process to obtain encapsulated THC, encapsulated CBD or encapsulated THC+CBD was carried out multiple times, with only slight deviations in the calculated encapsulation efficiencies. Thus, the present invention opens the way to a robust and quantitatively reproducible process of encapsulating one or more cannabinoids.

Yet another advantage of the process of the invention is that the produced particles contain virtually no residues of the solvent that is used for the extraction (see Examples' section 15). Thus, the solvent essentially completely migrates out of the droplets during the process. The solvent levels that were detected in the particles are well below the levels that are commonly imposed to commercial products, in particular to pharmaceutical products.

The cannabinoid component comprises one or more cannabinoids. In the context of the invention, a cannabinoid is understood to be a compound that naturally occurs in Cannabis plants such as Cannabis sativa, Cannabis indica or Cannabis ruderalis. Also included in cannabinoids are compounds that are derived from these naturally occurring compounds with retention of the characterizing terpenophenolic structural motif, for example by substitution of one or more functional groups. Typically, a cannabinoid has a structural motif that is derived from the structure of THC.

For example, the cannabinoid component comprises one or more cannabinoids selected from the group of delta-9-tetrahydrocannabinol, delta-8-tetrahydrocannabinol, cannabidiol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

Typically, the cannabinoid component (e.g. all the cannabinoids therein) has the property that, at the temperature at which the process of the invention is carried out, it does not form a single phase when it is mixed with the one or more compounds of the shell-forming component (i.e. with the one or more compounds selected from the group of C10-C30 fatty alcohols, C10-C30 fatty acids and esters of C10-C30 fatty alcohols and C10-C30 fatty acids) in a mass ratio of 50:50, e.g. when equal masses of both are mixed. In particular, the cannabinoid component has the property that it does not form a single phase with the one or more compounds when it is mixed with the one or more compounds in a mass ratio in the range of 60:40 to 40:60, in a mass ratio in the range of 70:30 to 30:70, in a mass ratio in the range of 80:20 to 20:80, in a mass ratio in the range of 10:90 to 90:10 or in a mass ratio in the range of 95:50 to 5:95.

The term ‘not forming a single phase’ includes the formation of two phases on the basis of non-miscibility (one phase does not dissolve in the other) as well as the formation of two phases on the basis of a different state of matter (e.g. a solid phase and a liquid phase).

Further, the definition that a particular first substance ‘does not form a single phase with a particular second substance at a particular temperature’ is to be viewed as a property of matter of the particular first substance as the neat substance (e.g. in pure form and not dissolved in any solvent), which property may be known from handbooks or which can be determined unambiguously by a person skilled in the art performing conventional procedures.

In an embodiment, at least 50 wt. % of the cannabinoid component consists of one or more cannabinoids. In another embodiment, this may be at least 60 wt. %, at least 70 wt. %, at least 80 wt. %, at least 90 wt. % or at least 95 wt. %. In particular, at least 98 wt. %, more in particular at least 99 wt. % and even more in particular at least 99.5 wt. % of the cannabinoid component consists of one or more cannabinoids. Non-cannabinoids in such cannabinoid component may for example be vitamins or terpenes. In case the cannabinoid component comprises a so-called whole-plant extract (vide infra), then the non-cannabinoids in the cannabinoid component are formed by other naturally occurring components of the Cannabis plant.

In a particular embodiment, the entire cannabinoid component consists of one or more cannabinoids.

In an embodiment, the one or more cannabinoids in the cannabinoid component comprises delta-9-tetrahydrocannabinol and one or more cannabinoids selected from the group of delta-8-tetrahydrocannabinol, cannabidiol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

In a particular embodiment, delta-9-tetrahydrocannabinol and cannabidiol are present in the cannabinoid component in a weight ratio in the range of 99.5:0.5 to 0.5:99.5, in the range of 99:1 to 1:99, in the range of 98:2 to 2:98, in the range of 97:3 to 3:97, in the range of 95:5 to 5:95, in the range of 90:10 to 10:90, in the range of 80:20 to 20:80, in the range of 30:70 to 70:30 or in the range of 40:60 to 60:40.

In another embodiment, the one or more cannabinoids in the cannabinoid component comprises cannabidiol and one or more cannabinoids selected from the group of delta-8-tetrahydrocannabinol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

In a particular embodiment, cannabidiol and cannabigerol are present in the cannabinoid component in a weight ratio in the range of 99.5:0.5 to 0.5:99.5, in the range of 99:1 to 1:99, in the range of 98:2 to 2:98, in the range of 97:3 to 3:97, in the range of 95:5 to 5:95, in the range of 90:10 to 10:90, in the range of 80:20 to 20:80, in the range of 30:70 to 70:30 or in the range of 40:60 to 60:40.

In an embodiment, the cannabinoid component comprises cannabidiol, cannabigerol and one or more fat-soluble vitamins selected from the group of vitamin A, vitamin D, vitamin E and vitamin K. In a particular embodiment, the one or more fat-soluble vitamins are vitamin K₂ and vitamin D₃.

In another embodiment, the delta-9-tetrahydrocannabinol is the main cannabinoid present in the cannabinoid component, i.e. more than 50 wt. % of the cannabinoid component consists of delta-9-tetrahydrocannabinol. For example, the content of delta-9-tetrahydrocannabinol is at least 60 wt. %, at least 75 wt. %, at least 85 wt. % or at least 90 wt. %. In particular, the content is at least 95 wt. %. More in particular, at least 99 wt. %, even more in particular at least 99.5 wt. %, yet even more in particular at least 99.9 wt. % of the cannabinoid component consists of delta-9-tetrahydrocannabinol.

In a method of the invention, the one or more cannabinoids in the solution may be obtained directly or indirectly by extraction from a Cannabis plant (e.g. delta-9-tetrahydrocannabinol may be obtained after extraction by decarboxylation of extracted tetrahydrocannabinolic acid). One or more particular cannabinoids may be isolated from the extract and then be used in the cannabinoid component in the solution. Alternatively, the entire extract may be used in the solution. In such case, the cannabinoid component comprises a so-called whole-plant cannabis extract.

The shell-forming component contains the material from which the shell is formed. The shell-forming component is mostly formed by the one or more compounds selected from the group of C10-C30 fatty alcohols, C10-C30 fatty acids and esters of C10-C30 fatty alcohols and C10-C30 fatty acids. Usually, the one or more compounds makes 100% of the weight of the shell-forming component, i.e. the shell-forming component consists of the one or more compounds. Optionally, the shell-forming component comprises one or more other compounds that also end up in the shell. For example, any such other compound(s) makes up 10 wt. % of the shell-forming component or less than that. It may also make up 7 wt. % or less, 5 wt. % or less, 3 wt. % or less, 2 wt. % or less, 1 wt. % or less or 0.5 wt. % or less. So, the one or more compounds of the shell-forming component makes up at least 90 wt. %, at least 93 wt. %, at least 95 wt. %, at least 97 wt. %, at least 98 wt. %, at least 99 wt. % or at least 99.5 wt. % of the shell-forming component.

The C10-C30 fatty alcohol may in principle be any fatty alcohol having from 10 to 30 carbon atoms. It is usually an alcohol selected from the group of capric alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, elaeostearyl alcohol, arachyl alcohol, gadoleyl alcohol, behenyl alcohol, erucyl alcohol and brassidyl alcohol.

It may also be any of the abovementioned fatty alcohols with a branched chain, as long as the total number of carbons does not exceed 30.

The C10-C30 fatty acid may in principle be any fatty acid having from 10 to 30 carbon atoms. It is usually an acid selected from the group of capric acid, lauric acid, palmitic acid, palmitoleic acid, stearic acid, isostearic acid, oleic acid, elaidic acid, petroselic acid, elaeostearic acid, arachic acid, gadoleic acid, behenic acid and erucic acid.

It may also be any of the abovementioned fatty acids with a branched chain, as long as the total number of carbons does not exceed 30.

The esters of C10-C30 fatty alcohols and C10-C30 fatty acids may also be composed of branched C10-C30 fatty alcohols and/or branched C10-C30 fatty acids.

In particular, the shell-forming component in the solvent comprises cetyl alcohol and/or cetyl palmitate.

In the process of the invention, droplets of the solution are generated in an aqueous medium. During the solvent migration, the droplets become the solid particles as the product of the process. The droplets are usually prepared in by conventional methods, in particular by stirring. Vigorous stirring is typically applied to obtain the droplets. A person skilled in the art can reach the appropriate conditions for this by routine experimentation and without exerting any inventive effort.

The temperature at which the process is performed (i.e. the operating temperature) may in principle be any temperature above the freezing point of water and below the boiling point of the solvent. For example, the temperature may be in the range 0-60° C., in the range of 5-50° C. or in the range of 10-35° C. Usually, however, the temperature is in the range of 15-30° C. In particular, it is in the range of 20-25° C.

In an embodiment, the process of the invention is followed by adding another substance to the solid particles resulting from the process. Such substance is generally an excipient, in particular a pharmaceutically acceptable excipient. The term excipient as used herein refers to any substance, generally pharmaceutically inert, used to formulate active pharmaceutical ingredients (API's-such as cannabinoids) into pharmaceutical formulations.

For example, an excipient is selected from the group of diluents, binders, glidants, lubricants, colouring agents, drying agents and disintegrants. The excipient material itself may be composed of one or more materials selected from the group of sugar alcohols, polyols (e.g. sorbitol, mannitol, xylitol), crystalline sugars, monosaccharides (e.g. glucose, arabinose), disaccharides (e.g. maltose, saccharose, dextrose, lactose), oligosaccharides (e.g. dextrins, cyclodextrins), polysaccharides (e.g. cellulose starch and derivatives thereof), inorganic salts (e.g. sodium chloride, calcium carbonate, magnesium carbonate, talc), and organic salts (e.g. sodium lactate, magnesium stearate).

A compound to be added may in particular be a coating agent. In such case, the process of the invention is usually followed by coating the solid particles resulting from the process with the coating agent. This then yields coated particles.

In yet another embodiment, the process of the invention is followed by preparing a pill or tablet from the solid particles resulting from the process. Preferably, such pill or tablet has a volume of 2 cm³ or less, more preferably of 1.5 cm³ or less and even more preferably of 1.0 cm³ or less.

The invention further relates to a cannabinoid-containing particle obtainable by the process described above.

A cannabinoid-containing particle that is obtained by a process of the invention has a core of one or more cannabinoids and a shell encapsulating this core. The shell compartmentalizes the one or more cannabinoids and so protects them against influences from the outer environment such as micro-organisms or reactive compounds such as oxygen.

Accordingly, the invention further relates to a cannabinoid-containing particle, wherein
one or more cannabinoids are encapsulated by a shell of one or more compounds selected from the group of C10-C30 fatty alcohols, C10-C30 fatty acids and esters of C10-C30 fatty alcohols and C10-C30 fatty acids;
the content of cannabinoid in the particle is in the range of 5-95 wt. %, in particular in the range of 25-85 wt. %.

For the cannabinoid-containing particle of the invention, the same considerations apply as those mentioned hereabove for the process of the invention, when the following is concerned: 1) the presence of the types of C10-C30 fatty alcohols; 2) the presence of the types of C10-C30 fatty acids; 3) the presence of the types of esters of C10-C30 fatty alcohols and C10-C30 fatty acids; 4) the presence of the types of cannabinoids.

Usually, the one or more cannabinoids has the property that it does not form a single phase with the one or more compounds of the shell when it is mixed with the one or more compounds of the shell in a mass ratio of 1:1 at 25° C., in particular a temperature in the range of 15-30° C., more in particular a temperature in the range of 0-50° C.

In particular, the one or more cannabinoids has the property that it does not form a single phase with the one or more compounds when it is mixed with the one or more compounds in a mass ratio in the range of 60:40 to 40:60, in a mass ratio in the range of 70:30 to 30:70, in a mass ratio in the range of 80:20 to 20:80, in a mass ratio in the range of 90:10 to 10:90 or in a mass ratio in the range of 95:5 to 5:95.

Usually, the content of cannabinoid in a particle of the invention is in the range of 20-80 wt. %, preferably it is in the range of 25-75 wt. %, more preferably it is in the range of 30-70 wt. % (the weight percentages are based on the total weight of the particle). Assuming no other constituents are present (in particular not encapsulated), it then follows that the shell constitutes 5-95 wt. % of the particle, in particular 15-75 wt. %. Usually, it is 20-80 wt. %, preferably 25-75 wt. %, more preferably 30-70 wt. %.

As noted earlier, a low or absent tackiness is likely the result of a shell that contains only small amounts of cannabinoid(s), or none at all, respectively. For example, particles with a THC content of 71 wt. % (and a shell content of 29 wt. %) were prepared (see SERIES A of the Examples, e.g. section 5). It was found that this product was dry and not tacky. This was also the case for particles comprising CBD (69 wt. %) and for particles comprising THC and CBD (72 wt. %). This is an indication that the shell (i.e. the substance of the shell itself) of a particle of the invention is substantially free of cannabinoid, in particular of delta-9-tetrahydrocannabinol. This means that the presence of cannabinoid in the particle is limited to the cannabinoid core of the particle. Similar results were obtained in SERIES B of the Examples, see e.g. section 13.

This is in contrast to many known cannabinoid-containing particles wherein cannabinoid is enclosed on the one hand, but on the other hand forms part of the enclosing material or is even localized onto such material (i.e. at the exterior of a particle at the interface with a surrounding atmosphere). This makes prior art particles sticky and the cannabinoid that they comprise subject to deterioration. For example, a wax that is present as a filler material forms an interpenetrating network between the cannabinoid(s). In particles with such composition, a plurality of cannabinoid domains is present and a significant portion of the cannabinoid is located at interfaces with a surrounding environment. The wax then remains tacky due to such morphology. In contrast, a particle of the invention in principle comprises only one domain, namely the core of the particle, which is separated from an outside environment all around.

It has been demonstrated that the cannabinoid in a particle of the invention is stable during extended periods of time. In SERIES A of the Examples, a period of at least 18 months is reported (section 6). In SERIES B of the Examples, a period of at least 6 months is reported (section 14). Such a high stability is (beside the non-tackyness) also an effect of the excellent shielding that is provided by the process of the invention. Moreover, particles of the invention have even proved to survive underwater storage for at least one month (see also section 14).

In a particle of the invention, the weight ratio of cannabinoid to shell is typically in the range of 0.25:0.75 to 0.95:0.05. It may also be 0.50:0.50 to 0.90:0.10, in the range of 0.40:0.60 to 0.80:0.20, or in the range of 0.60:0.40 to 0.95:0.05. This ratio is reflected by the ratio of cannabinoid component to shell-forming component in the solution that is used for preparing the particle.

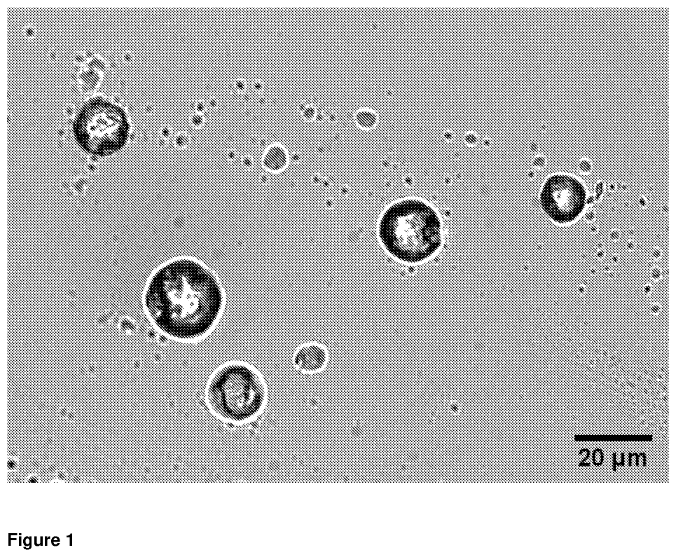

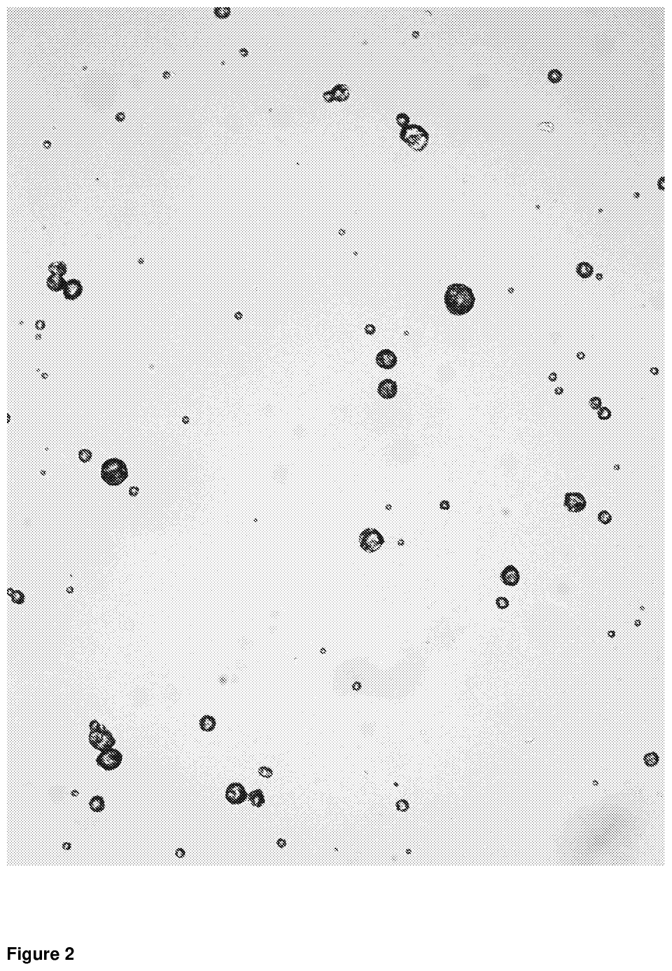

Cannabinoid-containing particles of the invention are displayed in the micrographs of FIG. 1 and FIG. 2, recorded with an optical microscope. The encapsulated cannabinoid is THC (FIG. 1) or CBD (FIG. 2). The particles in the micrographs are more or less spherical with a diameter of 10-20 μm. Their outer surface appears not completely smooth, as it appears to be wrinkled a little bit. The displayed particles are in an ambient atmosphere and do not stick to one another.

A cannabinoid-containing particle of the invention is usually globular. This shape is governed by the shape of the initial droplet in the process of the invention, which is usually globular.

The diameter of a particle of the invention is usually in the range of 100 nm-500 μm, in particular in the range of 250 nm-400 μm, more in particular in the range of 1-250 μm, even more in particular in the range of 3-100 μm or yet even more in particular in the range of 5-50 μm. For example, it is 200 nm or more, 300 nm or more, 400 nm or more, 500 nm or more, 750 nm or more, 1 μm or more, 2 μm or more, 3 μm or more, 4 μm or more, 5 μm or more, 6 μm or more, 7 μm or more, 8 μm or more, 9 μm or more, 10 μm or more, 12 μm or more, 14 μm or more, 20 μm or more, 30 μm or more, 40 μm or more, 50 μm or more, 75 μm or more, 100 μm or more, 200 μm or more, 300 μm or more, or 400 μm or more. It may also be 500 μm or less, 400 μm or less, 300 μm or less, 200 μm or less, 100 μm or less, 75 μm or less, 70 μm or less, 25 μm or less, 20 μm or less, 15 μm or less, 10 μm or less, 8 μm or less, 7 μm or less, 6 μm or less, 5 μm or less, 4 μm or less, 3 μm or less, 2 μm or less, 1 μm or less, 800 nm or less, 600 nm or less, 400 nm or less or 200 nm or less.

Usually, the one or more cannabinoids in a particle of the invention is selected from the group of delta-9-tetrahydrocannabinol, delta-8-tetrahydrocannabinol, cannabidiol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

In an embodiment, the one or more cannabinoids in a particle of the invention comprises delta-9-tetrahydrocannabinol and one or more cannabinoids selected from the group of delta-8-tetrahydrocannabinol, cannabidiol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

In a particular embodiment, delta-9-tetrahydrocannabinol and cannabidiol are present in a particle of the invention in a weight ratio in the range of 99.5:0.5 to 0.5:99.5, in the range of 99:1 to 1:99, in the range of 98:2 to 2:98, in the range of 97:3 to 3:97, in the range of 95:5 to 5:95, in the range of 90:10 to 10:90, in the range of 80:20 to 20:80, in the range of 30:70 to 70:30 or in the range of 40:60 to 60:40.

In another embodiment, the one or more cannabinoids in a particle of the invention comprises cannabidiol and one or more cannabinoids selected from the group of delta-8-tetrahydrocannabinol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

In a particular embodiment, cannabidiol and cannabigerol are present in a particle of the invention in a weight ratio in the range of 99.5:0.5 to 0.5:99.5, in the range of 99:1 to 1:99, in the range of 98:2 to 2:98, in the range of 97:3 to 3:97, in the range of 95:5 to 5:95, in the range of 90:10 to 10:90, in the range of 80:20 to 20:80, in the range of 30:70 to 70:30 or in the range of 40:60 to 60:40.

In a particular embodiment, a particle of the invention comprises cannabidiol, cannabigerol and one or more fat-soluble vitamins selected from the group of vitamin A, vitamin D, vitamin E and vitamin K. In a particular embodiment, the one or more fat-soluble vitamins are vitamin K₂ and vitamin D₃.

The shell of a particle of the invention is mostly composed of the one or more compounds selected from the group of C10-C30 fatty alcohols, C10-C30 fatty acids and esters of C10-C30 fatty alcohols and C10-C30 fatty acids. In a preferred embodiment, the one or more compounds makes 100% of the weight of the shell, i.e. the shell then consists of the one or more compounds. Optionally, the shell comprises one or more other compounds. For example, any such other compound(s) makes up 10 wt. % of the shell or less than that. It may also make up 7 wt. % or less, 5 wt. % or less, 3 wt. % or less, 2 wt. % or less, 1 wt. % or less or 0.5 wt. % or less. So, the one or more compounds of the shell makes up at least 90 wt. %, at least 93 wt. %, at least 95 wt. %, at least 97 wt. %, at least 98 wt. %, at least 99 wt. % or at least 99.5 wt. % of the shell.

The invention further relates to a cannabinoid-containing particle for use as a medicament.

The medical indications or uses for a cannabinoid-containing particle of the invention can broadly be broken down into the following categories: anti-nauseant and appetite stimulant, anti-spasmodic and anti-convulsant, analgesic (pain reliever), anti-inflammatory and immune system modulator, anxiolytic (anxiety reliever) and anti-depressant for mood disorders, and harm reduction substitute for alcohol, opiates, and other drugs.

More specifically, a cannabinoid-containing particle of the invention can be used in the treatment of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps. Further, cannabinoids have been reported to be effective in the treatment of diabetes, cancer, osteoporosis, sleep apneu, phantom limb, spinal/brain injury, Alzheimer's disease, glaucoma, neurodegenerative disease, Parkinson's disease, seizure, fatigue, post traumatic stress disorder, stress, organ rejection, ischemia, psoriasis, bone defects, AIDS, immunosuppressive disorder, muscular dystrophy, and vascular disorders.

For a particular use or treatment, such as any of the treatments mentioned above, the cannabinoid-containing particle of the invention is administered topically, orally, rectally or parenterally to a subject in need thereof. By topical administration of a medicament is meant that the medicament is directly applied to body surfaces such as the skin or mucous membranes. Application to other tissues than skin may involve the application of eye drops to the conjunctiva or ear drops placed in the ear. Topical administration may also be inhalational, such as asthma medications. The pharmacodynamic effect of topical administration may be local (at the application location) or systemic (transdermal application). In the latter case, the medicament is administered onto the skin but is absorbed into the body to attain systemic distribution. By parenteral administration of a medicament is meant that the medicament is injected directly into the body. Herein, the term injection encompasses intravenous injection, intramuscular injection, subcutaneous injection and intradermal injection.

For a particular use or treatment, such as any of the treatments mentioned above, the one or more cannabinoids in the cannabinoid-containing particle is administered at a dose in a range of 0.10-10 mg per kg of body weight to a subject in need thereof.

The invention further relates to a pharmaceutical composition comprising a cannabinoid-containing particle as described above and a pharmaceutically acceptable excipient.

The term excipient as used herein refers to any substance, generally pharmaceutically inert, used to formulate active pharmaceutical ingredients (API) into pharmaceutical formulations. For example, an excipient is selected from the group of diluents, binders, glidants, lubricants, colouring agents, drying agents and disintegrants. The excipient material itself may be composed of one or more materials selected from the group of sugar alcohols, polyols (e.g. sorbitol, mannitol, xylitol), crystalline sugars, monosaccharides (e.g. glucose, arabinose), disaccharides (e.g. maltose, saccharose, dextrose, lactose), oligosaccharides (e.g. dextrins, cyclodextrins), polysaccharides (e.g. cellulose starch and derivatives thereof), inorganic salts (e.g. sodium chloride, calcium carbonate, magnesium carbonate, talc), and organic salts (e.g. sodium lactate, magnesium stearate).

The invention further relates to a pharmaceutical composition as described above for use as a medicament. Such use in particular concerns the use as an anti-nauseant and appetite stimulant, as an anti-spasmodic and anti-convulsant, as an analgesic (pain reliever), as an anti-inflammatory and immune system modulator, as an anxiolytic (anxiety reliever) and anti-depressant for mood disorders, and as a harm reduction substitute for alcohol, opiates, and other drugs. More in particular, such use concerns the use in the treatment of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps. Further, cannabinoids have been reported to be effective in the treatment of diabetes, cancer, osteoporosis, sleep apneu, phantom limb, spinal/brain injury, Alzheimer's disease, glaucoma, neurodegenerative disease, Parkinson's disease, seizure, fatigue, post traumatic stress disorder, stress, organ rejection, ischemia, psoriasis, bone defects, AIDS, immunosuppressive disorder, muscular dystrophy, and vascular disorders.

For a particular use or treatment, such as any of the treatments mentioned above, the pharmaceutical composition of the invention is administered topically, orally, rectally or parenterally to a subject in need thereof.

For a particular use or treatment, such as any of the treatments mentioned above, the pharmaceutical composition is administered to a subject in need thereof in an amount wherein the cannabinoid dosage is in a range of 0.10-10 mg per kg of body weight.

For a particular use or treatment, such as any of the treatments mentioned above, the cannabinoid-containing particle is a delayed release composition for the release of one or more cannabinoids in for example the small intestine or the large intestine. In the context of the present invention, delayed release means that the composition releases the one or more encapsulated cannabinoids after passing through the stomach. In particular, a delayed release composition of the invention releases the one or more encapsulated cannabinoids in the distal small intestine. Preferably, no dissolution of the composition occurs in the stomach.

The invention further relates to a method for treating a medical condition of a human or an animal, comprising administering to the human or animal an effective amount of a cannabinoid-containing particle or a pharmaceutical composition as described above, wherein the medical condition is selected from the group of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

As used herein, the term “effective amount” means that amount of a drug or pharmaceutical agent that will elicit the biological or medical response of an animal or human that is being sought, for instance, by a researcher or clinician.

The invention further relates to the use of a cannabinoid-containing particle or a pharmaceutical composition as described above for treating a human or an animal.

The invention further relates to the use of a cannabinoid-containing particle or a pharmaceutical composition as described above for the manufacture of a medicament for the treatment of a medical condition, wherein the medical condition is selected from the group of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

The term medical condition as used herein is a broad term that includes all diseases, lesions, disorders, or nonpathologic condition that normally receives medical treatment, such as pregnancy or childbirth.

### EXAMPLES

Two series of experiments were performed wherein particles according to the invention were prepared. The first series was performed on a 4 mL scale (SERIES A). The second series, performed separately from the first series, was performed on a 1000 mL scale (SERIES B).

### Series A

### 1. Materials

The chemicals and solvents were purchased from commercial sources and used without further purification. If necessary, residual water was removed prior to use. When water was used, it was demineralized prior to use.

The following 10 mg/ml stock solutions were prepared:
1) cetyl alcohol in dichloromethane
2) cetyl palmitate in dichloromethane
3) THC in dichloromethane
4) THC+CBD in dichloromethane (50:50 wt. ratio)
5) CBD in dichloromethane
6) cetyl alcohol in benzyl alcohol
7) cetyl palmitate in benzyl alcohol
8) THC in benzyl alcohol
9) THC+CBD in benzyl alcohol (50:50 wt. ratio)
10) CBD in benzyl alcohol

The homogenizer that was used in the preparation of particles was an IKA T10 Basic Ultra Turrax® 0.0005-0.1 liter.

### 2. Preparation of Particles Using Dichloromethane

A 200 μl sample solution of 20 wt. % cetyl alcohol, 20 wt. % cetyl palmitate and 60 wt. % cannabinoid in dichloromethane was prepared by mixing:
40 μl of stock solution 1);
40 μl of stock solution 2);
120 μl of stock solution 3) or stock solution 4).

The sample solution thus had a solute content of 10 mg/ml. An Ultra Turrax® vial was charged with 3.96 mL of water which was then subjected to stirring at 28.000 rpm. During stirring, 0.04 mL of the sample solution was added to the vial. After the addition, stirring was continued for 5 minutes. The resulting mixture was then collected in a syringe and filtered over a syringe filter (PTFE/PES/CA). The residue on the filter was washed with 4 mL of water by pushing the water through the filter. After reversal of the syringe filter, the product particles were removed from the filter and collected in a vial by pushing 4 mL of water through the filter into the vial. The water was then removed by evaporation to yield a dry, white solid. No yield was determined for this composition. Instead, the amount of isolated (encapsulated) cannabinoid was determined, and compared to the theoretical maximum yield of encapsulated cannabinoid of 0.40 mg (as is described in section 4 herebelow).

Where THC and THC+CBD were encapsulated in a mixture of cetyl alcohol and cetyl palmitate, the CBD was encpasulated in only cetyl alcohol. Therefore, in the process of CBD, the sample solution was prepared from 80 μl of stock solution 1) and 120 μl of stock solution 5).

In all three cases (encapsulated THC, encapsulated THC+CBD and encapsulated CBD), it was observed that the isolated product was dry and not oily or tacky. Whereas compositions of cannabinoids, and in particular those comprising THC, are known to be tacky, these isolated products did not suffer from that. It was thus concluded that substantially all THC and/or CBD present in the product was not present at an interface with air, and was thus encapsulated.

### 3. Preparation of Particles Using Benzyl Alcohol

The same procedure was followed as the one using dichloromethane, albeit with a few modifications. The sample solution was prepared from stock solution 6), stock solution 7) and one of the stock solutions 8), 9) and 10). Further, the dissolution of cetyl palmitate in benzyl alcohol was aided by warming the mixture to 55° C. A micrograph of the isolated product from the encapsulation of THC is displayed in FIG. 1, showing a few separate particles. This forms direct evidence for the presence of the product of the invention in the form of particles. The particles obtained with the three encapsulation processes (THC, THC+CBD, CBD) are of a spherical form while their surface appears to have some relief. Their diameter is in the range of 10-20 μm.

### 4. Encapsulation Efficiency

The amount of THC and/or CBD that ends up in the isolated product was compared to the theoretical maximum value of 0.40 mg (encapsulation efficiency). This was performed for the product obtained with benzyl alcohol. To this end, the solid product obtained under section 3 hereabove was dissolved in acetonitrile and the resulting solution was quantitatively analyzed using Gas Chromatography. This was performed in triplicate. The encapsulation efficiency was then calculated by dividing the measured amount of THC in the final dry and solid product by the theoretical maximum.

The encapsulation efficiency of THC was 85% for the product obtained with benzyl alcohol (based on GC analysis and HPLC studies performed in triplicate).

The encapsulation efficiency of THC+CBD was 84% for the product obtained with benzyl alcohol (based on GC analysis and HPLC studies performed in triplicate).

The encapsulation efficiency of CBD was 81% for the product obtained with benzyl alcohol (based on GC analysis and HPLC studies performed in triplicate).

It should be stressed that the encapsulation efficiency concerns all the stages from the initial mixing of the stock solutions, via the Ultra Turrax® stirring en syringe filter washing, to the final drying of the product.

### 5. Particle Composition

The quantitative analysis of the particles as described in section 4 with Gas Chromatography also yielded the amounts of cetyl alcohol and cetyl palmitate in the particles. Accordingly, the weight percentage of THC in the product (and thus in the particles) could be calculated. The obtained values averaged to 71 wt. % of THC, 72 wt. % of THC+CBD and 69 wt. % of CBD.

### 6. Cannabinoid Stability Experiments

The stability of the cannabinoid contained in the particles prepared by the method of the invention was investigated by storing different samples of particles prepared as described in sections 2 and 3 hereabove, and analyzing them according to the procedure of section 4 hereabove. To this end, the samples were stored in a closed and dark container under atmospheric conditions. It appeared that no THC and/or no CBD deterioration could be observed with GC and HPLC during a period of as much as 18 months. Neither could a decrease of the THC and/or CBD quantity be observed during that period.

### Series B

### 7. Materials

The chemicals and solvents were purchased from commercial sources and used without further purification. Terpenes were extracted from hops. If necessary, residual water was removed prior to use. When water was used, it was demineralized prior to use.

The following 40 mg/ml encapsulation solutions were prepared, using dichloromethane as a solvent (mentioned ratios refer to weight percentages):
1. cannabidiol and cetyl alcohol 60:40
2. cannabidiol and cetyl alcohol 70:30
3. cannabidiol and cetyl alcohol 80:20
4. cannabidiol and cetyl palmitate 70:30
5. cannabidiol, cetyl palmitate and cetyl alcohol 60:20:20
6. cannabidiol, cetyl palmitate and behenyl alcohol 60:37.5:2.5
7. cannabidiol and palmitic acid 70:30
8. cannabidiol, nicotine, alpha-tocopherol and cetyl alcohol 23.3:23.3:23.3:30
9. cannabidiol, terpene extract and cetyl alcohol 50:20:30

The homogenizer that was used in the preparation of particles was an IKA T25 Digital Ultra Turrax® 0.05-2 liter.

All GC analyses were performed on an 8890 gas chromatograph (Agilent Technologies, Waldbronn, Germany) equipped with a flame ionization detector (FID) and an apolar column.

Micrographs of the isolated products were made using a Visiscope 500 microscope with 400× magnification factor.

### 8. Preparation of Terpene Extract

Dried flowers from Humulus (hops) were extracted using a Soxhlet set-up with dichloromethane as solvent. The extraction liquor was decolorized by five filtration steps over activated carbon. Dichloromethane was removed by rotational film evaporation to yield a light orange oil. The terpenes that are most abundantly present herein are humulene, caryophyllene, pinene and myrcene.

### 9. General Procedure for the Preparation of Particles

A 1.000 ml beaker charged with 792 mL of water was subjected to stirring at 3.000 rpm using the homogenizer. During the stirring, 8 mL of the encapsulation solution were added to the beaker. After the addition, stirring was continued for 1 minute. The resulting mixture was then subjected to an immersed air flow for 30 minutes to remove the dichloromethane from the water. The resulting suspension was filtered on a nylon filter membrane and washed with water to yield a solid. Any residual moist on the solid was removed by allowing the solid to dry to the air, yielding a dry, white powder.

### 10. General Procedure for the GC-Analysis of the Particles

The quantitative analysis of the particles was performed with gas chromatography (apparatus identified above in section 7). Helium was used as the carrier gas. The GC instrument conditions were as follows: start temperature 45° C.; gradual increase to 300° C.; total run time of 22 minutes.

Samples for the GC-analysis were prepared by dissolving a known mass of product (i.e. the produced particles) in a known volume of a suitable solvent. An amount of 1 μl of the sample solution was injected in the GC. The quantitative amounts of the components in the sample solutions were determined against standard solutions each containing a known amount of the component(s). The sample content is interpolated between two concentration levels in the standard solution.

### 11. Preparation of the Particles

The general procedure for the preparation of particles (see section 9 above) was performed for encapsulation solutions 1-9, yielding nine different products as dry, white solids.

### 12. Micrograph Analysis of the Different Products 1-9

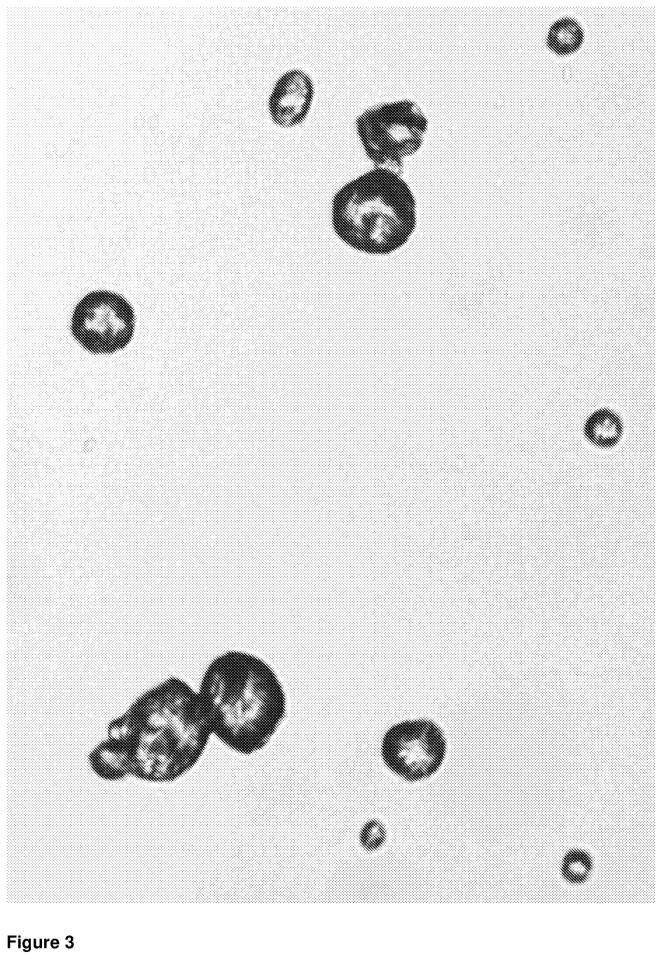

Micrographs of all obtained products were recorded. All micrographs display separate particles, which all have a similar appearance (i.e. they do not differ much from one micrograph to another micrograph). The micrograph of the isolated product resulting from encapsulation solution 6 (cannabidiol, cetyl palmitate and behenyl alcohol with mass ratio 24:15:1) is displayed in FIG. 2. An enlargement of the particles in the bottom left corner of FIG. 2 is displayed in FIG. 3. The product shown in FIGS. 2 and 3 is representative for the products obtained with the other encapsulation solutions, therefore only a micrograph of this product is provided. In all micrographs, the isolated product was visible as particles of a spherical form with some apparent relief on their surface. Their diameter is in the range of 10-20 μm. This forms direct evidence for the presence of the product of the invention in the form of particles.

### 13. GC-Analysis Results of the Different Products 1-9

The isolated products were analyzed by GC-analysis according to the general procedure described above (section 10). A quantitative analysis based on the obtained GC-data is shown in Table 1.

TABLE 1
Quantitative analysis of the products

| Sample | Relative | Encapsulated compound | | | | Shell | | | |
|---|---|---|---|---|---|---|---|---|---|
| No. | abundancies | CBD | NICO | ATOC | TERP | CA | CP | BA | PA |
| 1 | initial wt. % | 60 | | | | 40 | | | |
| | measured wt. % | 61 | | | | 41.2 | | | |
| 2 | initial wt. % | 70 | | | | 30 | | | |
| | measured wt. % | 69.3 | | | | 29.7 | | | |
| 3 | initial wt. % | 80 | | | | 20 | | | |
| | measured wt. % | 78.6 | | | | 20.9 | | | |
| 4 | initial wt. % | 70 | | | | | 30 | | |
| | measured wt. % | 69.7 | | | | | 30.9 | | |
| 5 | initial wt. % | 60 | | | | 20 | 20 | | |
| | measured wt. % | 59.7 | | | | 20.9 | 21.5 | | |
| 6 | initial wt. % | 60 | | | | | 37.5 | 2.5 | |
| | measured wt. % | 62.2 | | | | | 38.8 | 3.2 | |
| 7 | initial wt. % | 70 | | | | | | | 30 |
| | measured wt. % | 69 | | | | | | | 24.1 |
| 8 | initial wt. % | 23.3 | 23.3 | 23.3 | | 30 | | | |
| | measured wt. % | 29.2 | 1.2 | 28.9 | | 41.1 | | | |
| 9 | initial wt. % | 50 | | | 20 | 30 | | | |
| | measured wt. % | 57.1 | | | 4 | 28.7 | | | |

List of used abbreviations:

| | |
|---|---|
| CBD = cannabidiol | CA = cetyl alcohol |
| NICO = nicotine | CP = cetyl palmitate |
| ATOC = alpha-tocopherol | BA = behenyl alcohol |
| TERP = terpene extract | PA = palmitic acid |

The initial weight percentages (referring to the starting compounds) and the measured weight percentages (referring to the compounds that make up the particle) represent the weight percentages relative to the other compounds that are present. It was however also checked whether the measured weight percentages reflect the absolute weight percentages in the particles (which in theory may deviate due to degradation or contamination). It was found that the absolute weight percentages of the compounds in the particles corresponded nicely to the (relative) weight percentages that are presented in the table.

The table shows that the initial ratio of cannabinoid to shell-forming component in the sample solutions is largely maintained in the different products that were formed.

As regards the encapsulation of terpenes extracted from hop, it is noted that this extract concerns a mixture of terpenes wherein humulene, caryophyllene, pinene and myrcene are most abundant. There are however also other terpenes present in the extract, as well as some non-terpenes that were co-extracted form the hop. This likely explains why the measured wt. % exhibits a significant deviation from the initial wt. %.

The overall yield of encapsulated cannabinoid in the process, based on the initial amount added in the encapsulant solution, was at least 65% for the nine different products.

Analysis of the water phase of the particle preparation, aqueous filtration liquids, aqueous washing liquids and eventual other aqueous supernatants generated in the preparation process and sample preparation did not show the presence of cannabinoids and/or shell materials. It is contemplated that any product loss mainly occurs during filtration and washing procedures as described in section 9, for example because part of the product cannot be removed from a filter (dissolution of residual product on a filter would destroy the particles). Moreover, when filter residue is analysed, it exhibits the same ratio of cannabinoid component to shell-forming component. Therefore, the overall conversion to the encapsulated cannabinoid of the process may well be nearly 100%.

### 14. Cannabinoid Stability Experiments

The stability of the cannabinoid(s) contained in the particles was investigated by storing the different samples of particles. To this end, the samples were stored in an ICH standard climate cabinet with controlled temperature and moisture (−20° C., 5° C., 25° C., RH=65%). At certain time intervals, a product sample was taken and analyzed according to the procedures as described hereabove. Analysis of the particles demonstrated 100% retention of all cannabinoids after 6 months.

Also, particles were suspended in standard deionized water for up to one month. Afterwards, the particles showed 100% retention of all cannabinoids while no cannabinoids could be detected in the water phase. This indicates that particle degradation and cannabinoid leaking to the water does not occur during underwater storage of particles of the invention.

### 15. Determination of Residual Dichloromethane

It was determined whether the particles as prepared above comprise any residual dichloromethane. The quantitative analysis method used for this is the ‘method of standard addition’.

A sample solution was prepared by dissolving an accurately weighted amount of product sample in an appropriate solvent. Four different analyte solutions were prepared by dissolving different amounts dichloromethane in the appropriate solvent. The dichloromethane concentrations of the four analyte solutions were 0 μg/g, 300 μg/g, 600 μg/g and 900 μg/g. Four volumetric flasks were filled with equal and exactly determined volumes of the sample solution, which were then diluted to the same volume by adding one of the dichloromethane analyte solutions. The solutions were measured on the GC (column at 30° C. for 3 minutes followed by a gradual increase to 300° C.; total runtime of approx. 13 minutes) and the results were plotted; amounts of analyte added (x) against the signal (y). Extrapolation to the point on the x-axis at which y=0 corresponded to the amount of dichloromethane in the product sample.

All obtained values were well below the limit of quantification of the method (which is 40 μg/g). For example, for the product obtained with encapsulation solution No. 2 (cannabidiol and cetyl alcohol), the following three values were obtained: 4.5 μg/g; 11.9 μg/g; and 7.3 μg/g.

This proves that the dichloromethane that is used in the method for preparing the particles is virtually absent in the particles, and in any case is well below the levels that are commonly imposed to commercial products, in particular to pharmaceutical products.

1. Process for preparing cannabinoid-containing particles, comprising
providing a solution wherein the following components are dissolved in a solvent that has a water solubility in the range of 2-100 g/L at 25° C.:
a cannabinoid component comprising one or more cannabinoids;
a shell-forming component comprising one or more compounds selected from the group of C10-C30 fatty alcohols, C10-C30 fatty acids and esters of C10-C30 fatty alcohols and C10 C30 fatty acids;
generating droplets of the solution in an aqueous medium and allowing the solvent to migrate from the droplets to the aqueous medium to thereby form solid particles wherein a shell of shell-forming component encapsulates the cannabinoid component.

2. Process according to claim 1, wherein the process is followed by adding another substance to the solid particles, such as a pharmaceutically acceptable excipient.

3. Process according to claim 2, wherein the substance is selected from the group of diluents, binders, glidants, lubricants, colouring agents, drying agents and disintegrants.

4. Process according to claim 2, wherein the substance is selected from the group of sugar alcohols, polyols (e.g. sorbitol, mannitol, xylitol), crystalline sugars, monosaccharides (e.g. glucose, arabinose), disaccharides (e.g. maltose, saccharose, dextrose, lactose), oligosaccharides (e.g. dextrins, cyclodextrins), polysaccharides (e.g. cellulose starch and derivatives thereof), inorganic salts (e.g. sodium chloride, calcium carbonate, magnesium carbonate, talc), and organic salts (e.g. sodium lactate, magnesium stearate).

5. Process according to claim 1, wherein the process is followed by preparing a pill or tablet from the solid particles, wherein the pill or tablet has a volume of 2 cm³ or less.

6. Process according to claim 1, wherein the cannabinoid component comprises one or more cannabinoids selected from the group of delta-9-tetrahydrocannabinol, delta-8-tetrahydrocannabinol, cannabidiol, cannabinol, cannabigerol, tetrahydrocannabivarin, cannabidivarin, cannabichromene, delta-9-tetrahydrocannabinolic acid, delta-8-tetrahydrocannabinolic acid, cannabidiolic acid, cannabichromevarin, cannabigerovarin, cannabigerol monomethyl ether, cannabielsoin, cannabicitran, cannabicyclol and cannabivarin.

7. Process according to claim 1, wherein the cannabinoid component comprises a whole-plant cannabis extract.

8. Process according to claim 1, wherein the solvent is selected from the group of 1-butanol, n-butyl acetate, gamma-butyrolacton, chloroform, 1,2-dichloroethane, diethylene glycol, diethyl ether, diethoxyethane, di-isopropylether, dimethyl sulfoxide, ethyl acetate, methyl t-butyl ether, N-methyl-2-pyrrolidinone, nitromethane, 1-pentanol, 2-pentanol, 3-pentanol, 3-pentanone, benzaldehyde, prenol, o-cresol, m-cresol, and p-cresol.

9. Process according to claim 1, wherein the solvent is benzyl alcohol or methylene chloride.

10. Process according to claim 1, wherein the solvent has a solubility in water that is in the range of 8-80 g/L at 25° C., in particular in the range of 10-40 g/L at 25° C.

11. Process according to claim 1, wherein the shell-forming component comprises cetyl alcohol and/or cetyl palmitate.

12. Process according to claim 1, wherein the weight ratio of cannabinoid component to shell-forming component in the solution is in the range of 0.25:0.75 to 0.95:0.05, in particular in the range of 0.55:0.45 to 0.75:0.25.

13. Cannabinoid-containing particle obtainable by a process according to claim 1.

14. Cannabinoid-containing particle, wherein
one or more cannabinoids are encapsulated by a shell of one or more compounds selected from the group of C10-C30 fatty alcohols, C10-C30 fatty acids and esters of C10-C30 fatty alcohols and C10-C30 fatty acids;
the content of cannabinoid in the particle is in the range of 25-95 wt. %.

15. Cannabinoid-containing particle according to claim 13, wherein the shell is substantially free of cannabinoid.

16. Cannabinoid-containing particle according to claim 13, wherein the weight ratio of cannabinoid to shell is in the range of 0.50:0.50 to 0.90:0.10 or in the range of 0.60:0.40 to 0.95:0.05.

17. Cannabinoid-containing particle according to claim 13, wherein the particle has a diameter in the range of 100 nm-500 μm, in particular in the range of 1-50 μm.

18. Cannabinoid-containing particle according to claim 13 for use as a medicament.

19. Cannabinoid-containing particle according to claim 13 for use in the treatment of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

20. Cannabinoid-containing particle for use according to claim 18, wherein the particle is administered topically, orally, rectally or parenterally.

21. Cannabinoid-containing particle for use according to claim 18, wherein the cannabinoid-containing particle is administered in an amount wherein the dosage of the one or more cannabinoids is in a range of 0.10-10 mg per kg of body weight.

22. Pharmaceutical composition comprising
1) a cannabinoid-containing particle according to claim 13; and
2) a pharmaceutically acceptable excipient.

23. Pharmaceutical composition according to claim 22 for use as a medicament.

24. Pharmaceutical composition according to claim 22 for use.

25. Pharmaceutical composition for use according to claim 23, wherein the pharmaceutical composition is administered topically, orally, rectally or parenterally.

26. Pharmaceutical composition for use according to claim 23, wherein the pharmaceutical composition is administered in an amount wherein the dosage of the one or more cannabinoids is in a range of 0.10-10 mg per kg of body weight.

27. Pharmaceutical composition for use according to claim 23, wherein the composition is a delayed release composition for the release of one or more cannabinoids in the small intestine and/or the large intestine.

28. Method for treating a medical condition of a human or an animal, comprising administering to the human or animal an effective amount of a cannabinoid-containing particle according to claim 13 or of a pharmaceutical composition, wherein the medical condition is selected from the group of allergies, inflammations, infections, asthma, arthritis, cancer, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.
